(19) United States
(12) Patent Application Publication
(10) Pub. No.: US 2011/0236921 A1
(43) Pub. Date: Sep. 29, 2011

(54) CELL SYSTEM

(75) Inventors: ALBERT FRANCIS SANTOS, CARDIFF (GB); NICHOLAS THOMAS, CARDIFF (GB); ELIZABETH P. ROQUEMORE, CARDIFF (GB); CATHERINE HATHER, CARDIFF (GB)

(73) Assignee: GE HEALTHCARE UK LIMITED, LITTLE CHALFONT (GB)

(21) Appl. No.: 13/040,325

(22) Filed: Mar. 4, 2011

Related U.S. Application Data
(60) Provisional application No. 61/310,927, filed on Mar. 5, 2010.

(51) Int. Cl. C12Q 1/02

(52) U.S. Cl. 435/29

(57) ABSTRACT

The present invention relates to cellular systems for testing drug candidates and for evaluating the function of mitochondrial proteins. The invention is particularly useful for evaluating drug candidates for cancer and for conducting studies on drug resistance.

### CROSS-REFERENCE TO RELATED APPLICATIONS

This application claims priority to U.S. provisional patent application No. 61/310,927 filed Mar. 5, 2010; the disclosure of which is incorporated herein by reference in its entirety.

### FIELD OF THE INVENTION

The present invention relates to cellular systems and methods for testing drug candidates and for evaluating the function of mitochondrial proteins. The invention is particularly suited for studies on drug resistance.

### BACKGROUND OF THE INVENTION

Estimates from The American Cancer Society conclude that about 68,720 new melanomas will be diagnosed in the United States during 2009. Cancer of the skin is the most common of cancers, probably accounting for at least half of all cancers. Melanoma accounts for less than 5% of skin cancer cases but causes a large majority (75%) of skin cancer deaths (see the website for American Cancer Society).

A persistent challenge in developing effective therapies targeted at solid human tumors, including melanomas, is the acquisition of multi-drug resistance, and hence continued survival, of a sub-population of tumor cells. Many human solid tumors (>40%) are resistant to chemotherapy. Tumor cells acquire this survival trait as a result of changes in cellular mechanisms that can affect drug sensitivity and resistance, such as 1) alteration to cell-cycle patterns and metabolism, 2) reduced apoptosis, 3) increased DNA repair, and importantly, 4) the increased efflux of drugs.

There is therefore still a pressing need to develop better cell models and systems for improved drug screening and to find ways of understanding and overcoming drug-resistance.

The ability of any cell to pump out drugs (or toxins) is principally mediated by ATP-binding cassette (ABC) transporters. In normal cells, ABC transporters (there are 48 known human genes) bind and hydrolyze ATP and are involved in aspects of cellular homeostasis, including 1) the transport of heme, phospholipids and peptides, 2) the influx of essential nutrients and calcium, 3) the efflux of toxins. ABC transporter proteins are now also thought to play a key role in mediating cell survival by protection against oxidative stress.

Some ABC transporters are known to be predominantly localized on the inner surface of mitochondrial membranes, suggesting an important role for mitochondria in tumor cell resistance. Indeed, in many solid tumors, the increase in cellular ABC transporter activity is accompanied by changes to mitochondrial function and appearance, such as 1) lower mitochondria numbers, 2) high glycolysis rates, 3) smaller shape and size, 4) altered membrane composition, and 5) lowered membrane potential.

Recently, Elliot A. and Al-Hajj A. (ABCB8 mediates doxorubicin resistance in melanoma cells by protecting the mitochondrial genome. Mol Cancer Res. 2009; 7(1):79-87), showed that specific mitochondrial ABC transporter proteins confer doxorubicin (Adriamycin) resistance in human malignant melanoma cell lines. Doxorubicin, an anti-neoplastic drug, has found widespread use in the treatment of many human cancers, but it can be highly toxic and has poor efficacy for specific treatment of human malignant melanoma, which remains a major killer. In normal cells, doxorubicin causes damage to (non-histone-protected) mitochondrial DNA. Knockdown experiments with a range of shRNAs (short hairpin) interfering RNA (siRNA) targeting mitochondrial ABC proteins (including ABCB8, ABCC1, ABCC2, and ABCC5) sensitizes melanoma cells to doxorubicin treatment, leading to decreased cell viability.

Other mitochondrial proteins (superoxide dismutases, SOD) are known to play key roles in cell homeostasis and potential sensitivity of tumor cells to drugs (Hurt, E., et al., Integrated molecular profiling of SOD2 expression in multiple myeloma. Blood. 2007 May 1; 109(9): 3953-3962). SOD2 binds to the superoxide by-products of oxidative phosphorylation and converts them to hydrogen peroxide and diatomic oxygen, effectively protecting cells against ROS (reactive oxygen species) toxicity. SOD2 has also been implicated as a candidate tumor suppressor gene for human malignant melanoma, where observed down-regulation of transcription of SOD2 (by DNA methylation of the SOD2 promotor) appears to maintain tumor viability

Another mitochondrial protein, MTP18, serves as a downstream target for PI3K signaling, and is essential for cell viability. Loss-of-function of MTP18 results in mitochondrial morphology changes with accompanying cytochrome c release, leading to apoptotic cell death in normal cells. Targeting P13K signaling is an attractive intervention point for potential anti-cancer drugs.

In addition to specific biochemical molecular targets for anti-cancer therapy, there are distinct metabolic differences between tumor cells and the normal phenotype. In vivo, it was shown by Warburg in 1924 that many tumors survive and proliferate by circumventing oxidative phosphorylation in favor of glycolysis. These tumor cells show high rates of glucose uptake, increased glycolysis, and accumulation of lactic acid.

A key component in the armory for the screening for anti-cancer drugs is to employ immortalized tumor cells cultured in vitro. These latter cells are typically grown in classic culture conditions containing high glucose concentrations and abundant oxygen. Similar to those observations of tumor cells in vivo, although cells grown in vitro have a complement of fully competent mitochondria, they rely exclusively on glycolysis, rather than oxidative phosphorylation, to derive their ATP (known as the “Crabtree effect”).

As a result, cultured cells in vitro remain resistant to a number of drugs that would normally impair mitochondrial function, such as rotenone, antimycin, doxorubicin, oligomycin and tamoxifen. In turn, these cells potentially become more vulnerable to drugs that exploit the glycolytic dependency.

It has been shown that substitution of standard glucose-containing media for galactose leads to increased sensitivity of certain tumor cell types to mitochondrial toxicants, by shunting cells from their predominant anaerobic (glycolytic) respiration to oxidative phosphorylation. In particular, Marroquin, L. et al. (Circumventing the Crabtree effect: Replacing media glucose with galactose increases susceptibility of HepG2 cells to mitochondrial toxicants. Toxicol Sciences 2007; 97(2):539-547) found that replacement of glucose in the growth medium with galactose shunts hepatocyte cells (HepG2 line) towards the oxidative phosphorylation pathway in order to generate ATP, effectively bypassing the Crabtree effect. Consequently, these cells became more susceptible to the same drugs (exemplified above) that were previously shown to be ineffective in high glucose-containing media.

Targeting mitochondria and monitoring mitochondrial status is very important for the assessment of sensitivity and toxicity of potential therapeutic drugs in both normal and tumor cells. There is therefore a need to develop new cell based assays based upon tumor cells, which more closely mimic the in vivo situation, that can be used in cancer research and as screening tools in drug discovery. The present invention addresses this need and provides a system for testing drug candidates and for evaluating the function of mitochondrial proteins. The system can be used to observe synergistic or inhibitory effects on target cells.

### SUMMARY OF THE INVENTION

According to a first aspect of the invention, there is provided a method of preparing a cell system for testing a drug candidate or evaluating the function of a mitochondrial protein, comprising:

a) conditioning a first portion of a solid human tumor cell line to provide an oxidatively respiring cell culture;
b) conditioning a second portion of said solid human tumor cell line to provide an anaerobically respiring cell culture;
c) treating a first fraction of both said oxidatively respiring cell culture and said anaerobically respiring cell culture with an interfering RNA to produce two populations of cells having differentially functioning mitochondrial proteins therein; and
d) treating a second fraction of both the oxidatively respiring cell culture and the anaerobically respiring cell culture with a control set of interfering RNA species to produce two populations of cells having functional mitochondrial proteins therein.

In step c) one cell population will contain a mitochondrial protein which can be classified as “dysfunctional” and the other cell population will contain a mitochondrial protein which can be classified as “normal” in function,

Examples of an interfering RNA include, but are not limited to, small interfering RNA (siRNA) and a short hairpin RNA (shRNA), together with other forms of natural and synthetic molecules.

In a first aspect, step a) is carried out by growing said first portion in a galactose rich medium.

In a second aspect, step b) is carried out by growing said second portion in a glucose rich medium.

In another aspect, the solid human tumor cell line is a cancer cell line selected from the group consisting of breast cancer, prostate cancer, liver cancer, pancreatic cancer and skin cancer. The skin cancer cell line is a melanoma cell line selected from the group consisting of WM1552C, WM39 and WM115.

In one aspect the functional mitochondrial protein is selected from the group consisting of SOD2, MTP18 and ATP-binding cassette (ABC) transporters.

In a further aspect, the ABC transporter is selected from the group consisting of ABCB8, ABCC1 and ABCC2.

According to a second aspect of the present invention, there is provided a cell system prepared as hereinbefore described.

In a third aspect of the present invention, there is provided a method of testing a drug candidate using the cell system as hereinbefore described, comprising the steps of:

a) incubating a drug candidate with said populations of the oxidatively respiring cell culture having differentially functional mitochondrial proteins therein;
b) incubating said drug candidate with said populations of the anaerobically respiring cell culture having differentially functional mitochondrial proteins therein; and
c) determining any difference in the response of the populations of the oxidatively respiring cell culture and the populations of the anaerobically respiring cell culture to the drug candidate.

According to a fourth aspect of the present invention, there is provided a method of evaluating the function of a mitochondrial protein using the cell system as hereinbefore described, comprising the steps of:

a) growing said populations of the oxidatively respiring cell culture having differentially functional mitochondrial proteins therein;
b) growing said populations of the anaerobically respiring cell culture, having differentially functional mitochondrial proteins therein; and
c) determining any difference in the growth of the populations of the oxidatively respiring cell culture and the populations of the anaerobically respiring cell culture.

In one aspect, the difference in the response or growth of the cell cultures is determined by means of an imaging device or an activated cell sorter. A suitable imaging device may be, for example, the IN Cell Analyzer 2000 (GE Healthcare).

In a fifth aspect of the present invention, there is provided a use of the cell system as hereinbefore described for testing drug candidates or evaluating the function of a mitochondrial protein.

According to a sixth aspect of the present invention, there is provided a use of the cell system as hereinbefore described for testing for multi-drug resistance.

### BRIEF DESCRIPTION OF THE DRAWINGS

FIG. 1 shows a schematic representation illustrating conditioning of a WM115 cell line to an oxidatively respiring (WM115-Gal) or anaerobically respiring (WM115-Glu) cell culture, in accordance with the present invention.

FIG. 2 shows a graph illustrating that glucose-depleted cells (WM115-Gal) show increased oxidative respiration.

FIG. 3a and FIG. 3b show images of anaerobically (WM115-Glu) and oxidatively (WM115-Gal) respiring cells, respectively.

FIG. 3c shows a profile chart showing phenotypic differences between the cells of FIG. 3a and FIG. 3b.

FIG. 4a shows an image scan of a 384-well plate from a live-cell validation study where cells (WM115-Glu and WM115-Gal) have been treated with increasing concentrations of antimycin A.

FIG. 4b shows a graph reporting cell viability (as measured by cell number) when WM115-Glu and WM115-Gal cells are treated with the same drug.

FIG. 5a shows a phenotypic profile for WM115-Glu cells.

FIG. 5b shows a phenotypic profile for WM115-Gal cells.

FIG. 6 shows an image scan of a 384 well plate from a live-cell ABCB8 knockdown validation study showing differences between WM115-Gal and WM115-Glu cells.

FIG. 7a and FIG. 7b show images (with insets showing magnified cells) of WM115-Gal cells treated with Control (scrambled siRNA; a) and siRNA directed against ABCB8 (b).

### DETAILED DESCRIPTION OF THE INVENTION

### 1. Work-Flow

1.1 siRNA-Mediated Knockdown

Grow stock human melanoma cells, for example, but not limited to, WM793B, WM1552C, WM39, WM115, in 25 mM glucose-containing media (glycolytic activity) or 10 mM galactose-containing media (oxidative phosphorylation).

Plate out cells into 384-well microplates; Transfect cells with siRNA targeted against mitochondrial proteins. Reverse transfection protocol may be used, but not limited to, Lipofectamine L2K transfection reagent. Methods for carrying out reverse transfection are well documented in the literature. Choice of siRNA will include 1) the ABC proteins (ABCB8, ABCC1, ABCC2), 2) SOD2 and 3) MTP18.

Set up suitable controls (chemical and non-knockout siRNA).

Incubate for 24 h.

Stain cells (live or fixed) with (for example, but not limited to) combinations of Hoechst 33342, MITOTRACKER® Green, MITOTRACKER® Deep Red and MITOSOX™.

Image cells on imaging instrument, for example GE Healthcare IN Cell Analyzer 2000.

Analyze data for mitochondrial mass, morphology, membrane potential status and presence of superoxide radicals, cell number, nuclear integrity etc with for example, GE Healthcare IN Cell Investigator software.

1.2 siRNA-Mediated Knockdown in Combination with 1 or 2 Selected Drugs

Grow stock human melanoma cells, for example, but not limited to, WM793B, WM1552C, WM39, WM115, in 25 mM glucose-containing media (glycolytic activity) or 10 mM galactose-containing media (oxidative phosphorylation).

Plate out cells into 384-well microplates; Transfect cells with siRNA targeted against mitochondrial proteins. Reverse transfection protocol may be used and, but not limited to, Lipofectamine L2K transfection reagent. Methods for carrying out reverse transfection are well documented in the literature. Choice of siRNA will include 1) the ABC proteins (ABCB8, ABCC1, ABCC2), 2) SOD2 and 3) MTP18

Set up suitable controls (chemical and non-knockout siRNA)

Expose cells to drugs (including, but not limited to) antimycin, doxorubicin or tamoxifen (in DMSO or MeOH) (triplicate, 3 [final] doses of drug: 0.1 μM, 1 μM, 10 μM)+zero drug control.

Incubate defined time period (24 h).

Stain cells (live or fixed) with (for example, but not limited to) combinations of Hoechst 33342, MITOTRACKER® Green, MITOTRACKER® Deep Red and MITOSOX™.

Image cells on imaging instrument, for example IN Cell Analyzer 2000 (GE Healthcare).

Analyze data for mitochondrial mass, morphology, membrane potential status and presence of superoxide radicals, cell number, nuclear integrity etc with Image analysis software, for example, GE Healthcare IN Cell Investigator software.

### 2. Experimental and Results

### 2.1 Conditioning of WM-115 Human Melanoma Cells Into Glucose and Glucose-Free (Galactose) Medium

The starting cell line, WM-115 Human melanoma (ECACC Cat 91061232 #04/H/007) was cultured in complete growth medium—Minimum Essential medium Eagle (MEM, Sigma M2279 containing 5 mM glucose), adding 50 ml foetal bovine serum (10%), 5 mL glutamine (2 mM), 5 mL penicillin/streptomycin, 5 mL non essential amino acids (1%), 5 mL sodium pyruvate (1%).

Cells were maintained in culture for approximately 2 weeks before conditioning process was started. High glucose (25 mM) and glucose-free medium was then prepared:

1. High Glucose medium (Glu-med)—DMEM (Invitrogen, 21969 containing 25 mM glucose, 1 mM sodium phosphate), adding 50 ml FBS, 2.5 ml HEPES (5 mM) and antibiotics.
2. Glucose-free medium (Gal-med)—DMEM (Invitrogen 11966, containing no glucose), adding 50 mL FBS, 2.5 mL HEPES (5 mM), 5 mL pen/strep, 5 mL sodium pyruvate (1 mM), 5 mL glutamine (2 mM, in addition to 4 mM in base medium), 10 mL galactose solution (10 mM).

Cells were cultured in a 50% mix of either Glu-med with MEM or Gal-med with MEM for 3 days. Cells continued to grow well therefore were transferred to a 75% mix of Glu- or Gal-media with MEM for a further 3 days. Cells continued to grow and were transferred to 100% Glu- or Gal-medium.

After three days, cell growth had slowed considerably in both media. The phenotype of cells in glucose-free medium (Gal-med) altered from the WM-115 parental cell type. Gal-med cells appeared larger, flatter and less elongated. After further 3 days, Glu-med cells also altered and appeared smaller and less elongated than the parental WM-115 cells.

After a further three days, the growth of both cell types improved sufficiently for cells to require passaging. Viability was assessed as >92% for both cell types and cells were maintained in culture for a further 3 weeks (approximately 10 passages) before generating frozen stocks and performing characterization.

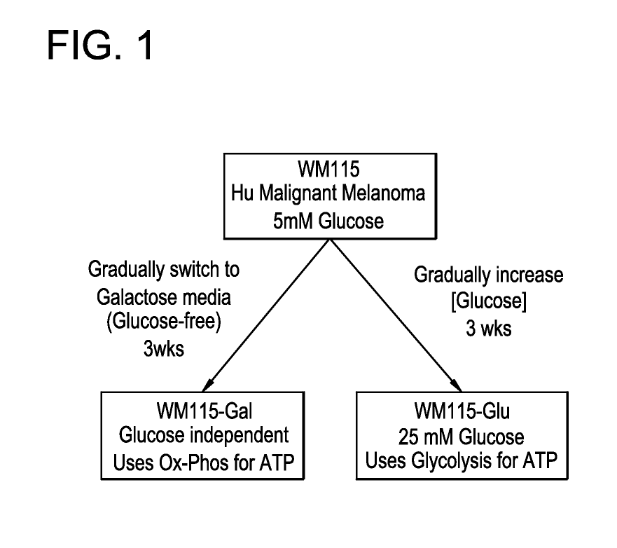

FIG. 1 shows a schematic workflow of a parental human melanoma cell line (WM115) that was re-conditioned to glucose-free or glucose-rich culture medium to produce respective daughter cell lines dependent on either mitochondrial oxidative phosphorylation (WM115-Gal) or glycolysis (WM115-Glu), as described above.

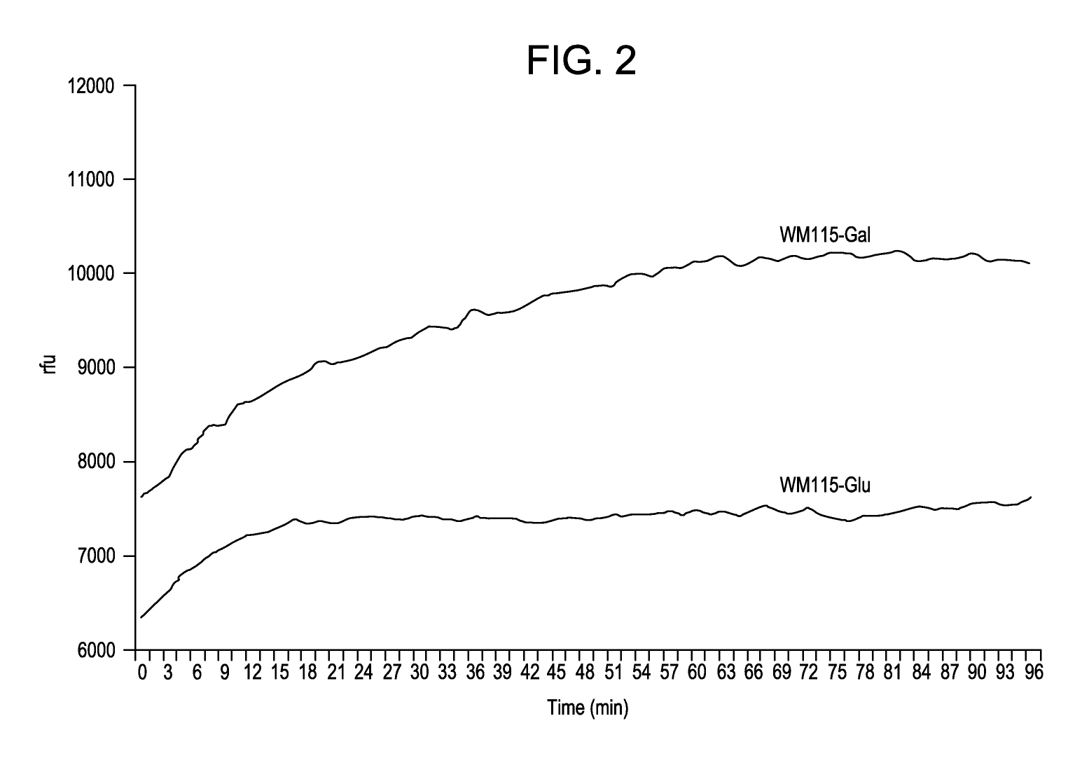

A cell-impermeable, phosphorescent, oxygen sensitive probe (MITOXPRESS™ from Luxcel) was used to measure the extra-cellular oxygen concentration (FIG. 2). The assay is based on the ability of oxygen to quench the excited state of the probe. As the cell population respires, oxygen is depleted in the surrounding environment, which results in un-quenching of the probe and therefore a signal increase. Changes in oxygen consumption reflecting changes in mitochondrial activity are seen as changes in probe signal over time.

Probe signal is measured using a standard fluorescence plate reader and a 96-well microtitre plate. Results show that the galactose-conditioned cells do indeed have an increased rate of respiration compared to the cells adapted to glucose-rich culture medium (probe phosphorescence increases over time for the galactose-adapted cells but remains fairly constant for those grown in the glucose-rich environment).

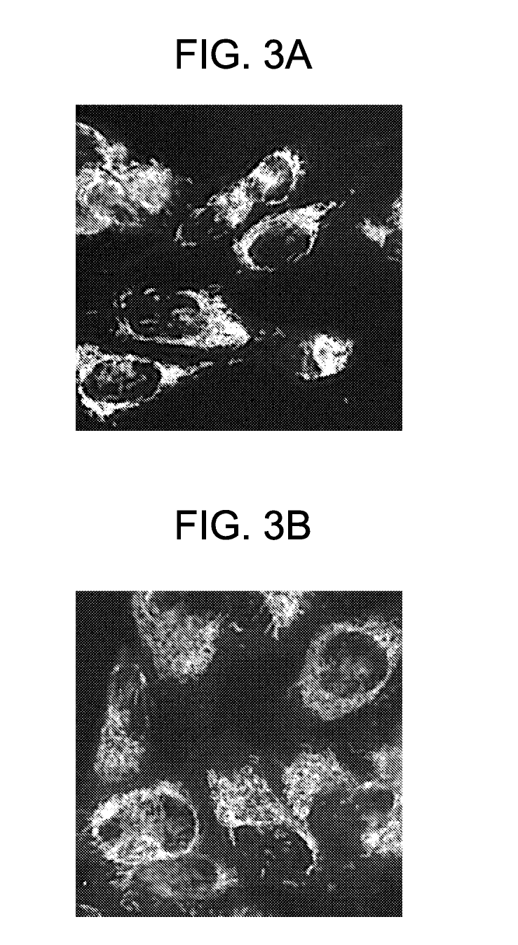

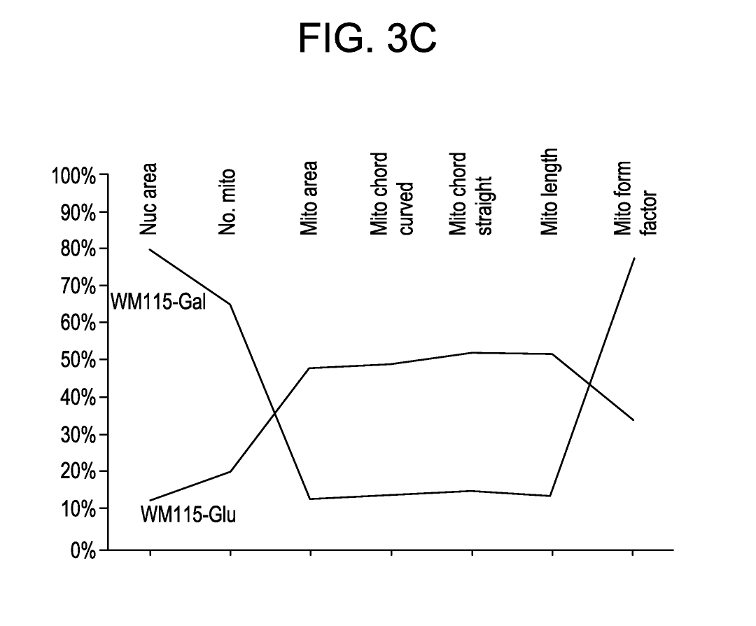

FIG. 3a and FIG. 3b are images of anaerobically (WM115-Glu) and oxidatively (WM115-Gal) respiring cells, respectively, while FIG. 3c is a profile chart showing morphological phenotypic differences between these cells. The differentially conditioned cell lines have distinct phenotypic appearances and profiles. Mitochondria were stained with MITOTRACKER® Red CMXRos and Hoechst 33342 (Invitrogen). Images of live cells were acquired on IN Cell Analyzer 2000 (40×/0.6NA objective). Morphology differences for the glucose-deprived (WM-Gal) cells (FIG. 3b) include larger nuclei and a more extensive mitochondrial network (in agreement with increased dependence on mitochondria and increased respiration). Automated analysis demonstrates distinct multi-parameter phenotypes; the profile chart in FIG. 3c (created using Spotfire, which is linked within GE Healthcare IN Cell Investigator software) shows the clear phenotype differences for the WM115-Gal cells compared to the WM115-Glu cells (for a range of measures).

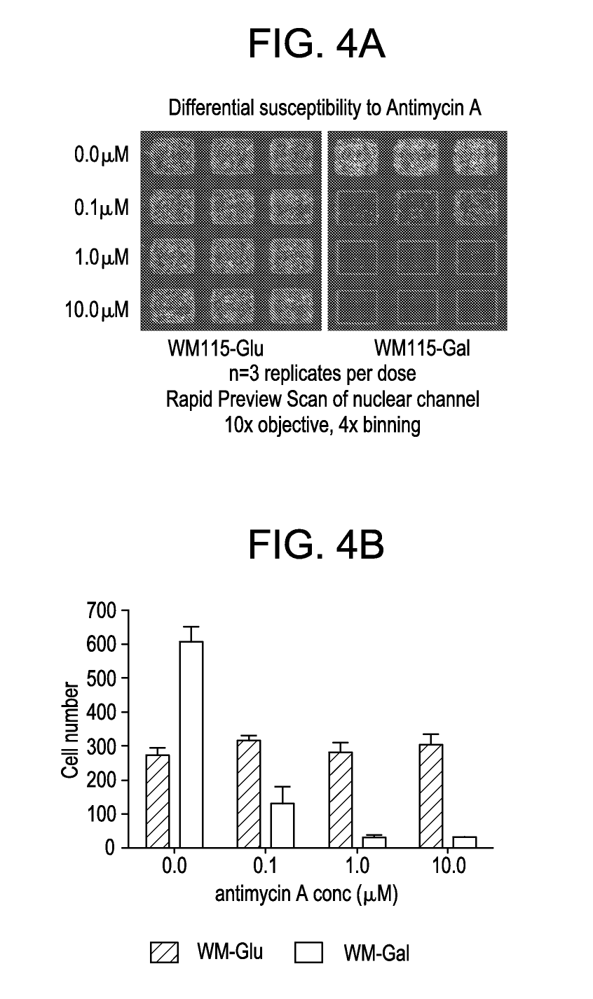

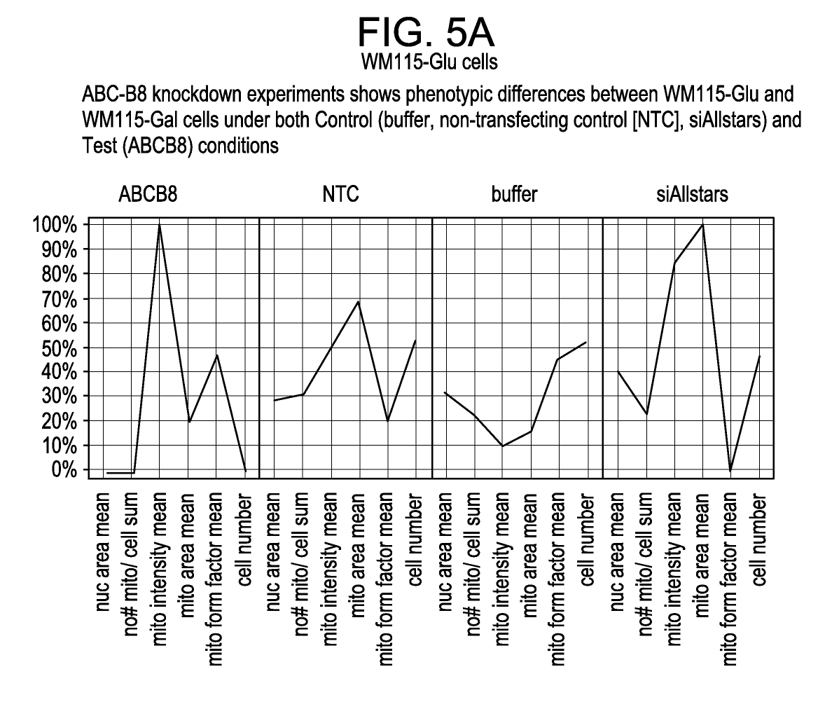

FIG. 4a shows an image scan in the nuclear channel of a 384-well live-cell validation plate revealing that that the glycolytic cell line (WM115-Glu) is resistant to antimycin A (tested at 10.0 μM) whereas the respiring cell line (WM115-Gal) is susceptible to antimycin A (as low as 0.1 μM). Live cells are clearly visible in the full concentration range for WM115-Glu cells, but not for WM115-Gal cells (low cell density in 0.1 μM→10.0 μM antimycin A). FIG. 4b is a graphical representation of the same data.

Antimycin A acts on respiration complex II of the electron transport chain. Glucose-deprived cells (WM115-Gal) are dependent on mitochondrial respiration for ATP, and are therefore sensitive to antimycin A. In contrast, the WM115-Glu cells grown in glucose-rich media are reliant on glycolysis for ATP generation and as predicted, these cells appear resistant to antimycin A.

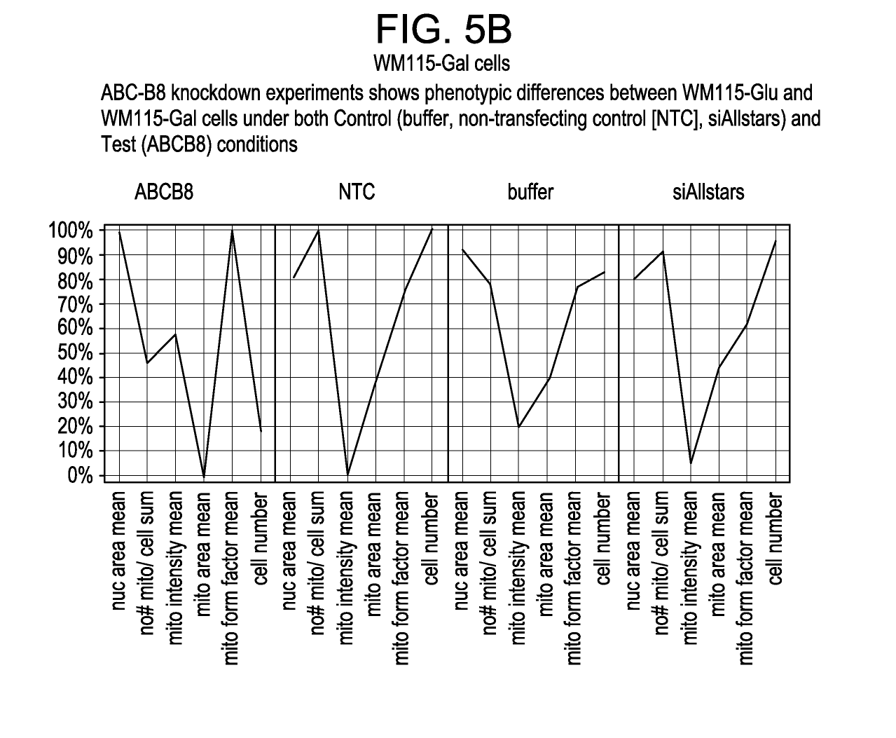

FIG. 5a and FIG. 5b depict example results extracted from a 384-well live-cell validation study. In the example shown, WM115-Glu and WM115-Gal cells were treated with both Control (buffer or non-transfecting control [NTC] or scrambled siRNA [AllStars] as well as Test conditions (siRNA directed against ABCB8) followed by staining and imaging as described earlier. From image analysis, the phenotypic profiles WM115-Glu and WM115-Gal show distinct differences for parameters such as nuclear count, mitochondrial area/number and mitochondrial stain intensity

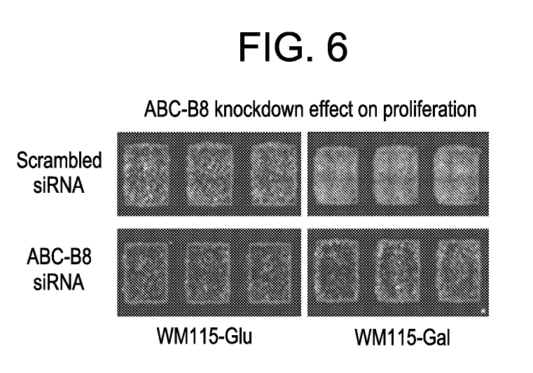

The image scans shown in FIG. 6 show the effect of knockdown of the mitochondrial ATP binding cassette protein ABCB8 for both cell types.

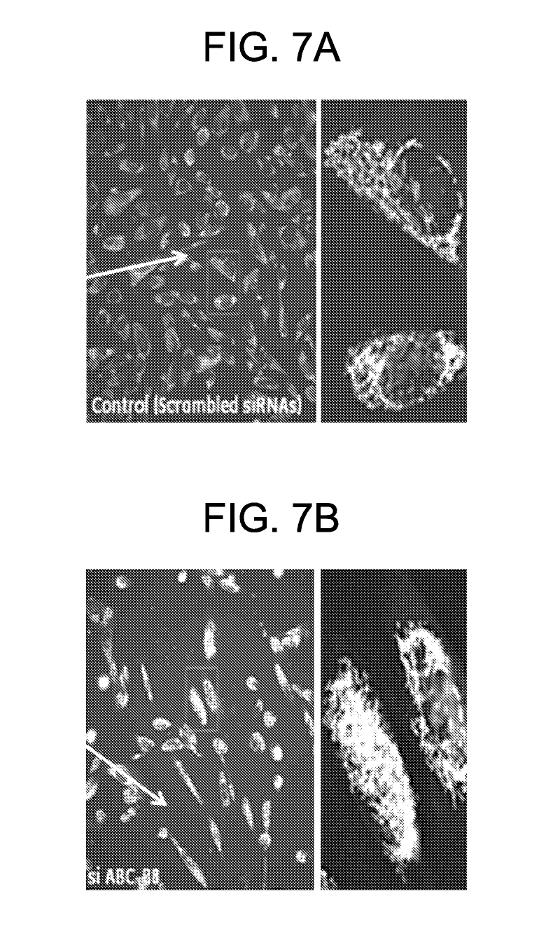

FIG. 7a and FIG. 7b are images (with insets showing magnified cells) of WM115-Gal cells treated with Control (scrambled siRNA; a) and siRNA directed against ABCB8 (b).

Although its function is largely unknown, ABCB8 is expressed in most human tissues. Some evidence suggests that yeast deficient in the ABCB8 homologue MDL2 has a decreased growth on glycerol-containing or oleate-containing media compared to wild-type cells, suggesting a role for the protein in regulation of mitochondrial lipid homeostasis.

Another line of evidence indicates that ABCB8 forms a complex with other mitochondrial proteins, and that this complex is involved in protection of cells against oxidative stress.

The data indicate that melanoma cells (growing in glucose-rich conditions that mimic the tumor environment) may be susceptible to intervention at ABCB8-dependent processes.

Whilst the present invention has been described in accordance with various aspects and preferred embodiments, it is to be understood that the scope of the invention is not considered to be limited solely thereto and that it is the Applicants' intention that all variants and equivalents thereof also fall within the scope of the appended claims.

What is claimed is:

1. A method of preparing a cell system for testing a drug candidate or evaluating the function of a mitochondrial protein, comprising:
a) conditioning a first portion of a solid human tumor cell line to provide an oxidatively respiring cell culture;
b) conditioning a second portion of said solid human tumor cell line to provide an anaerobically respiring cell culture;
c) treating a first fraction of both said oxidatively respiring cell culture and said anaerobically respiring cell culture with an interfering RNA to produce two populations of cells having differentially functional mitochondrial proteins therein; and
d) treating a second fraction of both the oxidatively respiring cell culture and the anaerobically respiring cell culture with a control set of interfering RNA species to produce two populations of cells having functional mitochondrial proteins therein.

2. The method of claim 1, wherein step a) is carried out by growing said first portion in a galactose rich medium.

3. The method of claim 1, wherein step b) is carried out by growing said second portion in a glucose rich medium.

4. The method of claim 1, wherein said solid human tumor cell line is a cancer cell line selected from the group consisting of breast cancer, prostate cancer, liver cancer, pancreatic cancer and skin cancer.

5. The method of claim 4, wherein said skin cancer cell line is a melanoma cell line selected from the group consisting of WM1552C, WM39 and WM115.

6. The method of claim 1, wherein said mitochondrial protein is selected from the group consisting of SOD2, MTP18 and ATP-binding cassette (ABC) transporters.

7. The method of claim 6, wherein said ABC transporter is selected from the group consisting of ABCB8, ABCC1 and ABCC2.

8. A cell system prepared by the method of claim 1.

9. A method of testing a drug candidate using the cell system of claim 8, comprising the steps of:
a) incubating a drug candidate with said populations of the oxidatively respiring cell culture having differentially functional mitochondrial proteins therein;
b) incubating said drug candidate with said populations of the anaerobically respiring cell culture having differentially functional mitochondrial proteins therein; and
c) determining any difference in the response of the populations of the oxidatively respiring cell culture and the populations of the anaerobically respiring cell culture to the drug candidate.

10. A method of evaluating the function of a mitochondrial protein using the cell system of claim 8, comprising the steps of:
a) growing said populations of the oxidatively respiring cell culture having differentially functional mitochondrial proteins therein;
b) growing said populations of the anaerobically respiring cell culture, having differentially functional mitochondrial proteins therein; and
c) determining any difference in the growth of the populations of the oxidatively respiring cell culture and the populations of the anaerobically respiring cell culture.

11. The method of claim 9, wherein any said difference in the response or growth of the cell cultures is determined by means of an imaging device or an activated cell sorter.

12. The method of claim 10, wherein any said difference in the response or growth of the cell cultures is determined by means of an imaging device or an activated cell sorter.
